(19) United States
(12) Patent Application Publication
(10) Pub. No.: US 2016/0228262 A1
(43) Pub. Date: Aug. 11, 2016

(54) AN INSTRUMENT FOR POSITIONING A CUP COMPONENT OF AN ORTHOPAEDIC JOINT PROSTHESIS

(71) Applicant: Andrew BAILEY, Leeds (GB)

(72) Inventor: ANDREW BAILEY, LEEDS (GB)

(21) Appl. No.: 15/024,411

(22) PCT Filed: Sep. 29, 2014
(86) PCT No.: PCT/GB2014/052928

§ 371 Date: Mar. 24, 2016

(30) Foreign Application Priority Data
Sep. 30, 2013 (GB) 1317285.3

(51) Int. Cl. A61F 2/46; A61F 2/34

(52) U.S. Cl. CPC A61F 2/4609; A61F 2/34; A61F 2002/4623

(57) ABSTRACT

An instrument (102) for positioning a cup component (104) of an orthopaedic joint prosthesis is described. The instrument includes an actuator (124) and a plurality of jaw members (152, 154, 156, 158) around a longitudinal axis. Each jaw member includes a mounting end (162), a gripping end (166) and a surface (174) between them. The gripping end has a transversely extending member (168) which can be received in a recess in a wall of a cup component and can displaced between a retracted position and a deployed position. The surface is inclined to the longitudinal axis of the instrument and curved in an outward direction away from the longitudinal axis. The actuator and the plurality of jaw members are capable of relative movement along the longitudinal axis of the instrument with the actuator in contact with the inclined surfaces of the jaw members so that said relative movement causes the jaw members to move between their retracted position and deployed position.

This invention relates to an instrument for positioning a cup component of an orthopaedic joint prosthesis.

Certain orthopaedic joint prostheses include a hollow cup with an inner surface which defines a generally hemispherical hollow region, and another component which has a spherical part which can be received in the hollow region for articulation relative to the cup component. Such joint prostheses can include hip joint prostheses and shoulder joint prostheses. The exterior of the cup will contact the prepared surface of the patient's bone in which the component is to be implanted. The interior of the cup will present a smooth bearing surface to the spherical part of the other component of the joint prosthesis. The bearing surface can be provided by a single piece cup component. Alternatively, the cup component can comprise a shell part which contacts the prepared surface of the patient's bone, and a bearing part which provides the bearing surface, and which fits into the shell part. The bearing part can be made from a material which is different from the material of the shell part: for example the bearing part can be made from a polymeric material (such as polyethylene) or a ceramic material and the shell part (and the spherical part of the other component) can be made from a metal (such as a cobalt-chromium based alloy, or a stainless steel, or a titanium based alloy).

It is important that the components of an orthopaedic joint prosthesis are positioned accurately in a patient's bone. Both location and alignment are important. Accurate positioning of a component requires that the component be engaged by an appropriate instrument, allowing considerable force to be applied to the component if and as necessary. However, it can be important not to contact the external surface or the internal surface or both of the component with the instrument, especially the internal surface when it has been provided with a smooth polished bearing surface. Scratching or otherwise damaging that surface can impair the bearing properties of the prosthesis.

U.S. Pat. No. 5,171,243 discloses an acetabular cup for use in a hip joint prosthesis. The cup comprises a shell which has a circumferential groove cut into its inner surface. The groove can received a flange at the free end of an insertion tool so that the cup is retained on the instrument, allowing the shell to be manipulated using the instrument. The grooved shell part receives a bearing part which has a smooth inner surface against which a bearing surface of another component of the joint prosthesis can articulate. The shell part can have fastening holes extending through its wall through which bone screws can extend to fasten the shell part to the surface of a bone.

WO-A-2008/099242 discloses an instrument for gripping a cup component of an orthopaedic joint prosthesis which includes a plurality of jaw members which extend radially from a central drive shaft. The jaw members can be made to slide radially inwardly so that they engage the outside wall of a cup component. Each of the jaw members has a pin at one end which is received in a spiral track on a drive plate. The jaw members are made to slide radially by rotating the drive plate.

The present invention provides an instrument for positioning a cup component of an orthopaedic joint prosthesis, which includes a plurality of jaw members each having an inclined face, and an actuator which can slide longitudinally in contact with the inclined faces of the jaw members to cause them to move between deployed and retracted positions.

An aspect of the invention provides an instrument for positioning a cup component of an orthopaedic joint prosthesis, comprising an actuator and a plurality of jaw members arranged around a longitudinal axis of the instrument. Each jaw member can have a mounting end and a gripping end with a transversely extending member which can be received in a recess in a wall of a cup component. The gripping end of each of the jaw members can be displaced between a retracted position and a deployed position in which the gripping end is displaced from the retracted position in the direction in which the transversely extending member is directed. Each jaw member can further include a surface between the mounting end and the gripping end which is inclined to the longitudinal axis of the instrument, and wherein the actuator and the plurality of jaw members are capable of relative movement along the longitudinal axis of the instrument with the actuator in contact with the inclined surfaces of the jaw members so that said relative movement causes the jaw members to move between their retracted position and deployed position.

The instrument of the invention has the advantage that the number of parts from which it is made is small. Furthermore, assembly of the instrument from its parts can be simple. These advantages mean that the instrument can be manufactured simply and at low cost and is easy to clean.

The surface between the mounting end and the gripping end of each jaw member may be curved in an outward direction away from the longitudinal axis. The surface between the mounting end and gripping end may transition as a smooth and/or continuous curve extending from the mounting end to the gripping end. The surface between the mounting end and the gripping end of each jaw member may also be curved in a peripheral direction around the longitudinal axis.

The gripping end of each jaw member may include a flat underside surface arranged to engage an upper surface of a rim of the cup component. The flat underside surfaces may all be perpendicular to the longitudinal axis of the instrument. Each flat underside surfaces may be positioned between a respective transversely extending member and the longitudinal axis of the instrument. The flat underside surfaces may extend in a peripheral direction sufficiently to form a complete annular underside surface when the jaw members are in the deployed position.

The transversely extending members at the gripping ends of the jaw members can be directed inwardly. The transversely extending members can then be received in one or more recesses which are provided in an outwardly facing side wall of the cup component. Optionally, the actuator can be a hollow sleeve and the jaw members can extend within the actuator. The inclined surfaces of the jaw members can then be outwardly facing surfaces of the jaw members which contact an inwardly facing surface of the actuator.

When the transversely extending members at the gripping ends of the jaw members are directed inwardly, so that they can then be received in one or more recesses which are provided in the outwardly facing side wall of the cup component, the jaw members can have a surface towards the gripping ends of the jaw members which faces generally along the axis of the instrument, towards a cup component which is engaged by the fingers. The surface can act against a rim of the cup component so that an impaction force that is applied to the instrument is transmitted to the cup component.

The actuator can be in the form of a hollow cylinder. The cylinder can have a circumferentially continuous wall, although the invention can be implemented using an actuator in the form of a cylinder which has slots formed in its wall. The cylinder might be short so that it might be viewed as a collar. The jaw members can be arranged within the bore of an actuator which is in the form of a hollow cylinder.

The transversely extending members at the gripping ends of the jaw members can be directed outwardly. The transversely extending members can then be received in one or more recesses which are provided in an inwardly facing side wall of the cup component. Optionally, the jaw members can be located around the actuator and the inclined surfaces of the jaw members can then be inwardly facing surfaces of the jaw members which contact an outwardly facing surface of the actuator.

The actuator can be in the form of a rod having a cylindrical outer surface. The rod might be short so that it might be viewed as a cylindrical plug. The rod can be located within a hollow space which is defined by and between the jaw members, so that it and the jaw members are capable of relative movement along the longitudinal axis.

Optionally, the jaw members are biased resiliently towards their retracted positions. In an instrument which has this feature, the actuator can be used to apply a force to the jaw member against the bias, to cause them to move towards their deployed positions. The jaw members can move towards their retracted positions by moving the actuator and allowing the jaw members to move as a result of the biasing force. This can be a preferred arrangement because it means that the cup component is engaged positively by the instrument.

The jaw members can be biased resiliently towards their deployed positions. In an instrument which has this feature, the actuator can be used to apply a force to the jaw member against the bias, to cause them to move towards their retracted positions. The jaw members can move towards their deployed positions by moving the actuator and allowing the jaw members to move as a result of the biasing force.

Optionally, the jaw members can be provided by a component which flares outwardly towards the gripping ends of the jaw members. The component can have generally conical side walls. The inclined surfaces of the jaw members can be curved when the jaw members are viewed tangentially so that, for example, the component can be approximately bell-shaped when viewed from the side. The jaw members can be formed in the component by slots in the wall of the component which are open at the gripping end of the jaw members. The slots can extend from the gripping ends of the jaw members towards but not as far as the top of the component. The flared side walls of the component can be acted on by a hollow actuator which acts on the outwardly facing surfaces of the side walls. The flared side walls of the component can be acted on by an actuator which is located inside the housing and acts on the inwardly facing surfaces of the side walls.

The slots between adjacent jaw members can be relatively narrow. For example, when the cup component is circular so that the jaw members form a circle, it can be preferred that the ratio of the angle subtended at the axis of the instrument by each jaw member to the angle subtended by a slot between two adjacent jaw members is at least about 3, more preferably at least about 4.

The transversely extending member on each of the jaw members can have the same width as the jaw member itself so that the transversely extending member on each of the jaw members subtends the same angle at the axis of the instrument as the jaw member on which it is located.

The transversely extending member on each of the jaw members can be narrower than the jaw member so that the angle subtended by the transversely extending member on each of the jaw members at the axis of the instrument is smaller than the angle subtended by the jaw member on which it is located. For example, when the cup component is circular so that the jaw members form a circle, the ratio of the angle subtended at the axis of the instrument by the transversely extending member on each jaw member to the angle subtended by the jaw member can be less than about 0.5, or less than 0.3, for example less than 0.15.

The component can comprise a tube whose wall is flared outwardly and has slots formed in it at one end.

Optionally, the instrument includes a handle. Optionally, the jaw members are attached to the handle, and the actuator can be moved relative to the handle and the jaw members.

The instrument can have an impaction surface through which an impaction force can be applied to the instrument and to a cup component which is attached to the instrument. The instrument can include a central shaft which is in communication with the impaction surface to transfer the impaction force to the cup component. This can be used to force the cup component into tight fitting engagement with a prepared bone cavity.

Optionally, the instrument includes an actuator mechanism for causing the actuator to move relative to the handle and to the jaw members. The actuator mechanism can include a driver which is mounted so that rotation of the driver causes displacement of the actuator along the longitudinal axis of the instrument relative to the handle. The driver can have a sloped or tilted end face and the actuator can have a sloped or tilted end face against which the driver end face abuts. The degree of tilt or slope of the end faces of the driver and actuator relative to the longitudinal axis of the instrument can be the same.

The actuator mechanism can have other forms. For example, the actuator mechanism can include a camming arrangement, a threaded arrangement, a pin travelling in a helical slot, a wedge or a simple push action.

The instrument can have two jaw members. Preferably, the instrument has more than two jaw members. A preferred embodiment has three or four jaw members. The instrument can have up to ten jaw members.

Preferably the jaw members are arranged so that they are spaced apart uniformly around a cup component with which they are to be used. For example, when the instrument has three jaw members and the cup component is circular, the angle subtended between adjacent jaw members at the longitudinal axis of the instrument can be 120°. When the instrument has four jaw members and the cup component is circular, the angle subtended between adjacent jaw members at the longitudinal axis of the instrument can be 90°.

Component parts of the instrument can be made from polymeric materials or metallic materials or from both polymeric and metallic materials. Suitable polymeric materials include polyolefins, polyesters, engineering polymers such as polyether-ether-iketones, and acetals. Suitable metals include certain stainless steels, and titanium and its alloys. The use of polymeric materials for at least some of the component parts of the instrument has the advantage of lower cost and ease of manufacture, for example by moulding.

For many applications, the cup component will be rotationally symmetrical about a polar axis. It will often be preferred that the cup component has the shape of a portion of a sphere. As is known, the rim of a cup component to be fitted in a patient's acetabulum in a hip replacement procedure will frequently subtend an angle of at least 140° at the centre of the sphere, for example at least 150° or at least 160° or at least 170°. The rim of an acetabular cup component will frequently be circular, although sometimes the rim can have an extension on one side for example to reduce the risk of dislocation. When the rim of the cup component is circular, the rim can be planar around at least 50% of the periphery of the cup component. The plane defined by the rim can be perpendicular to the polar axis of the component.

The glenoid component of a shoulder prosthesis will generally be more shallow than the cup component of a hip prosthesis and its rim might not be circular.

The bone facing surface of the cup component can be adapted for contact with bone tissue. For example, it can be adapted to bond to the bone tissue as a result of direction interaction (for example bone ingrowth) of bone into the surface of the component, or it can be adapted to bond to the tissue by means of a bone cement material.

The cup component can have a plurality of discrete recesses arranged around its periphery in which the transversely extending members on the jaw members can be received. When the cup component has discrete recesses arranged around its periphery, the number of recesses should not be less than the number of jaw members. Frequently, the number of recesses and the spacing between the recesses should correspond to the number of transversely extending members and the spacing between the fingers.

The cup component can have a recess which is longer than each of the transversely extending members which are received in it, for example in the form of a groove which extends around at least part of the cup component. The groove can extend continuously around the entire periphery of the cup component.

The invention also provides an assembly for use in a surgical procedure to replace an orthopaedic joint, which comprises an instrument according to the preceding aspect of the invention, and a cup component of an orthopaedic joint prosthesis, in which each of the generally transversely extending members on the jaw members of the instrument are received in a recess in the wall of the cup component.

Embodiments of the invention are described below in detail, by way of example only, and with reference to the accompanying drawings, in which:

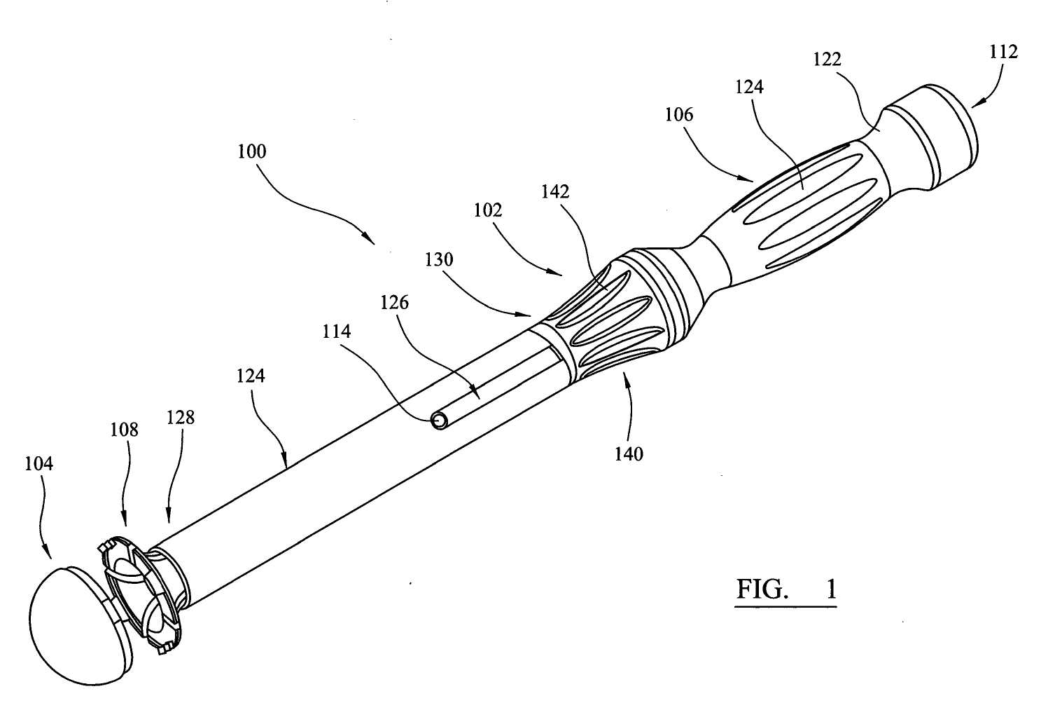

FIG. 1 is an isometric view of a cup component and an instrument for positioning the cup component according to the invention;

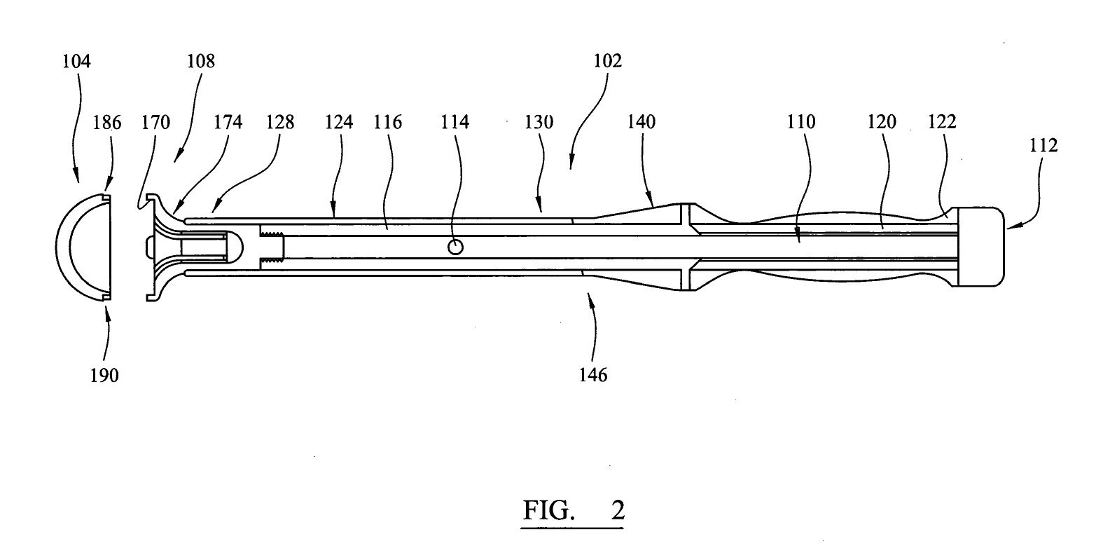

FIG. 2 is a cross sectional side view of the cup component and the instrument shown in FIG. 1;

FIG. 3 is an isometric view of an actuator mechanism part of the instrument shown in FIGS. 1 and 2;

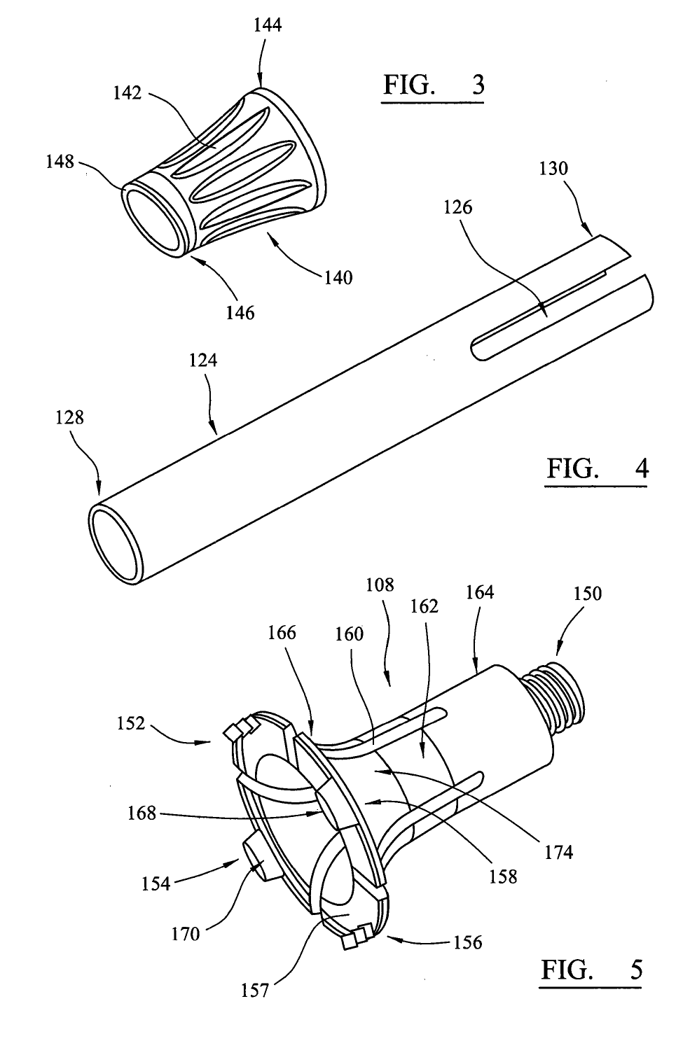

FIG. 4 is an isometric view of an actuator part of the instrument shown in FIGS. 1 and 2;

FIG. 5 is an isometric view of a cup holding part of the instrument shown in FIGS. 1 and 2;

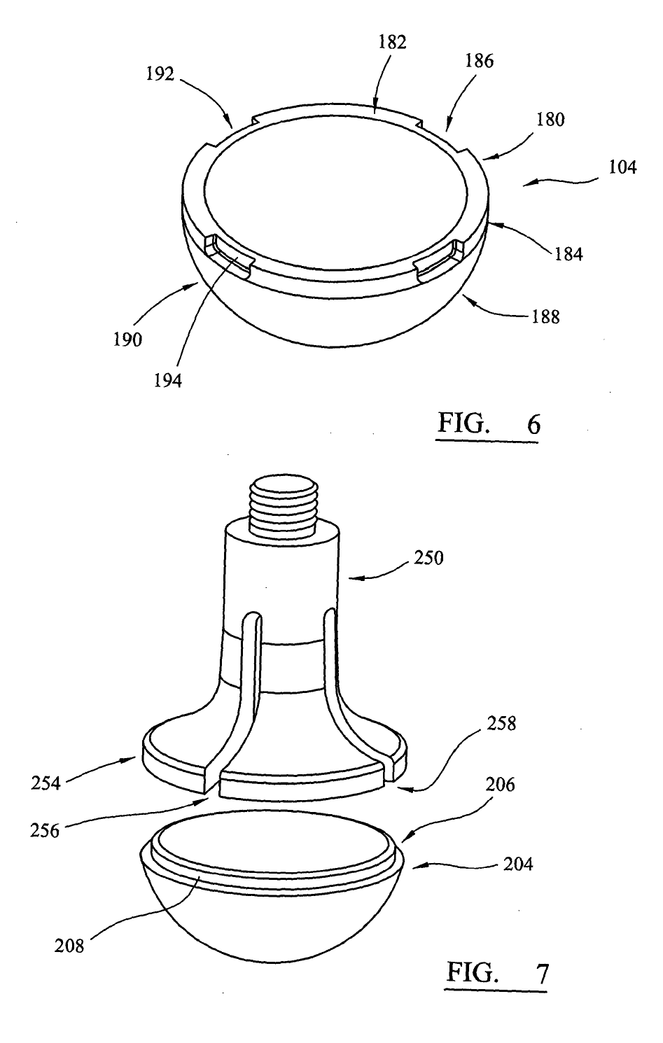

FIG. 6 is an isometric view of the cup component shown in FIGS. 1 and 2; and

FIG. 7 is an isometric view of a further embodiment of a cup holder part of the instrument and a cup component.

With reference to FIG. 1 there is shown an isometric view of an instrument 102 for positioning a cup component 104 of an orthopaedic joint prosthesis. The instrument 102 and cup 104 can be combined to form an assembly 100. FIG. 2 shows a transverse cross section along a longitudinal axis of the instrument 102 and cup component 104.

The instrument 102 includes a handle 106 at a proximal end, and a cup holding part 108 at a distal end. As best illustrated in FIG. 2, the instrument 102 has a central shaft 110 extending along its longitudinal axis and terminating at the proximal end in an impaction head 112. A first peg 114 and a second peg (not shown) extend transversely along its longitudinal axis from the central shaft. A sleeve 116 of a metal or engineering polymer surrounds shaft 110. A distal end of sleeve 116 defines a blind threaded bore by which the cup holding component 108 is attached to the instrument.

A further sleeve 120 of a metal or an engineering polymer surrounds a more proximal part of shaft 110 and handle 106 is formed by a grip 122 surrounding the shaft. The grip 122 may be formed of any suitable material, such as a silicone rubber or an engineering polymer. The grip 122 can include a plurality of longitudinal recesses, e.g. recess 124, to provide an enhanced grip for the handle part 106 of the instrument.

An actuator is provided between the handle 106 and cup holding component 108. The actuator includes a cylindrical tube 124 having a longitudinally extending closed slot, e.g. slot 126, in each side and for accepting each of the pair of pins, 114, which act to prevent rotation of the tube 124. A distal end 128 of push shaft 124 encircles and surrounds the cup holding component 108. A proximal end 130 of the push shaft 124 has end surfaces which are tilted relative to the longitudinal axis of the instrument in that they do not subtend an angle of 90° relative to the longitudinal axis of the instrument.

A rotatable collar 140 provides an actuator mechanism and is journalled about sleeve 116 between the push shaft 124 and handle 106. Collar 140 includes a number of longitudinal recesses, e.g. 142, in its outer surface to provide grip formations. The collar 140 is generally tubular and tapers from a greater outer diameter at a proximal end to a lesser outer diameter at a distal end 146. Distal end 146 provides an end surface 148 defining a plane which is tilted relative to the longitudinal axis of the instrument, in that it subtends an angle different to 90° and is not perpendicular to the longitudinal axis. The end face 148 of the collar subtends the same angle as the end face of the push shaft such that they are parallel, as illustrated in FIG. 1. The angle subtended by the end face of the collar/end face of the push shaft depends on the force needed to hold the cup.

The cup holding component 108 is shown in isolation in FIG. 5 and generally has the form of a flared, rotationally symmetric cylinder. A threaded boss 150 extends from a proximal end. The cup holding component 108 includes four jaw members 152, 154, 156, 158. Each jaw member 152, 154, 156, 158 is defined by a pair of slits, e.g. slit 160 extending through the wall of the component. Each slit can be a few mm wide, for example about 1 mm wide. Each jaw member includes a mounting end, e.g. 162 by which the jaw member includes a mounting end, e.g. 162 by which the jaw member is mounted to the main body 164 of the component. Each jaw member also includes a gripping end 166 which bears a member, 168 arranged to engage a corresponding recess in the cup component 104. Each member 168 extends at least partially in a direction transverse to the longitudinal axis of the instrument and presents an inwardly facing slightly sloped surface, e.g. 170. Each jaw member includes a surface, e.g. 174 extending between the gripping end and the mounting end and which is inclined relative to the longitudinal axis of the instrument. As best illustrated in FIGS. 1 and 2, an inner surface of the distal end 128 of the actuator 124 engages the inclined surface 174 and can be urged there over when the actuator 124 is translated along the longitudinal axis of the instrument as described in greater detail below.

FIG. 6 shows a perspective view of the cup component 104. As illustrated in FIG. 6, the cup 104 has a generally hemispherical shape and includes a rim portion 180 with an upper surface 182 and a peripheral side surface 184. Four recesses 186, 188, 190, 192 and a peripheral side surface 184. Four recesses 186, 188, 190, 192 are formed in an outer surface of the rim portion at equi-angularly spaced positions. Each recess, 186, 188, 190, 192 is positioned, sized and spaced to accept a respective one of the transverse extending members 152, 154, 156, 158. An inner surface e.g. 194 tilts inwardly slightly to form a recess into which the transversely extending member can be received and to abut against the inner wall 170 of each transversely extending member.

Operation of the instrument will now be described. FIG. 1 shows the jaw members in a retracted position in which the transversely extending members 152, 154, 156, 158 can be introduced into a respective recess 186, 188, 190, 192 in the rim of the cup component 104. The cup holding member 108 is made from a plastic material and is resiliently biased so that the jaw members are naturally held in the retracted position. In order to securely lock the cup component to the instrument 102, a user can operate the actuator mechanism by rotating collar 140 about the longitudinal axis of the instrument. The tilted end surface of the collar 140 acts against the tilted end surfaces of the proximal portion 130 of the push shaft. The pair of pegs engaged in the pair of slots in the push shaft constrain the push shaft to prevent its rotation such that the push shaft is caused to translate along the longitudinal axis of the instrument and toward the cup holding component 108. The distal end 128 of the push shaft 124 drives against the inclined surface 174 of each jaw member and causes the gripping ends of each jaw member to be displaced in a generally transverse direction into a deployed position in which the transversely extending members are urged into the recesses in the cup component and into abutment with the inner surfaces of each recess, e.g. 194. As the inner surface of each transversely extending member and recess are tilted slightly relative to the longitudinal axis, this provides a recess and tooth arrangement which prevents the cup from being released from the instrument in a longitudinal direction.

The instrument can then be used to manipulate the cup component into position and an impactor can be used to impart an impaction force on the cup instrument by impacting impaction head 112 of the instrument. As the impaction head is attached to the central shaft 110 of the instrument, this helps to transfer the impaction force along the instrument and on to the upper surface of the rim of the cup 182 via the flat underside surfaces, e.g. 157, of the gripping end of each jaw member.

In order to remove the instrument from the cup component 104, the actuator mechanism is rotated in the opposite sense which allows the actuator shaft 124 to travel along the longitudinal axis by the action of the inclined surfaces of each jaw, being resiliently biased toward the retracted position, acting on the distal end 128 of the actuator shaft 124. Hence, the jaw members can retain the original retractor position in which the transversely extending members are no longer received within the recesses in the cup and so the instrument can be removed from the cup component.

FIG. 7 shows a further embodiment of the cup holding component 250 and cup component 204. The cup holding component 250 is similar in construction to that shown in FIG. 5 other than the form of the transversely extending members. In the second embodiment of the cup holding member 250, each transversely extending member, 252, 254, 256, 258 extends along the entire peripheral length of the gripping end of each jaw member. The cup component 204 includes a single recess 206 in the rim of the cup component and extending around the entire periphery of the cup component 204.

Hence, in the retracted position, as illustrated in FIG. 7, the transversely extending members, which have a generally arcuate shape, can all be introduced into the same single recess 206 and the relative angular position of the cup and cup holding component 250 does not need to be carefully controlled. The actuator mechanism, when operated, causes the transversely extending members to move toward the deployed position in which an inner surface of the transversely extending members abuts an inner surface 208 of recess 206. Inner surface 208 is inclined slightly relative to the longitudinal axis as is an inner surface of the transversely extending members so as to prevent the cup component becoming detached by movement in a longitudinal direction.

Operation of the instrument and its actuation are largely described above. All that is varied are the details of the mechanism by which the cup component can be releasably and securely attached to the cup holding component of the instrument.

1. An instrument for positioning a cup component of an orthopaedic joint prosthesis, comprising:
an actuator; and
a plurality of jaw members arranged around a longitudinal axis of the instrument, each jaw member having:
a mounting end,
a gripping end with a transversely extending member which can be received in a recess in a wall of a cup component, wherein the gripping end of each of the jaw members can be displaced between a retracted position and a deployed position in which the gripping end is displaced from the retracted position in the direction in which the transversely extending member is directed, and
a surface between the mounting end and the gripping end which is inclined to the longitudinal axis of the instrument and curved in an outward direction away from the longitudinal axis, wherein the actuator and the plurality of jaw members are capable of relative movement along the longitudinal axis of the instrument with the actuator in contact with the inclined surfaces of the jaw members so that said relative movement causes the jaw members to move between their retracted position and deployed position.

2. The instrument of claim 1, wherein the surface extends as a continuous curve between the mounting end and the gripping end.

3. The instrument of claim 1, wherein the gripping end of each jaw member includes a flat underside surface arranged to engage an upper surface of a rim of the cup component.

4. The instrument of claim 3, wherein the flat underside surfaces are all perpendicular to the longitudinal axis of the instrument.

5. The instrument of claim 3, wherein each flat underside surfaces is positioned between a respective transversely extending member and the longitudinal axis of the instrument.

6. The instrument of claim 3, wherein the flat underside surfaces extend in a peripheral direction sufficiently to form a complete annular underside surface when the jaw members are in the deployed position.

7. The instrument of claim 1, in which the transversely extending members are directed inwardly.

8. The instrument of claim 7, in which the actuator is a hollow sleeve and the jaw members extend within the actuator, the inclined surfaces of the jaw members being outwardly facing surfaces of the jaw members which contact an inwardly facing surface of the actuator.

9. The instrument of claim 1, in which the transversely extending members are directed outwardly.

10. The instrument of claim 9, in which the jaw members are located around the actuator and the inclined surfaces of the jaw members are inwardly facing surfaces of the jaw members which contact an outwardly facing surface of the actuator.

11. The instrument of claim 1, in which the jaw members are biased resiliently towards their retracted positions.

12. The instrument of claim 1, in which each transversely extending member extends along substantially the entire peripheral length of each respective gripping end.

13. The instrument of claim 1, in which the jaw members are provided by a component having a wall which flares outwardly towards the gripping ends of the jaw members, and having slits in the wall, each slit being open at the gripping ends of the jaw members.

14. The instrument of claim 1, and including a handle.

15. The instrument of claim 14, in which the jaw members are attached to the handle, and in which the actuator can be moved relative to the handle and the jaw members.

16. The instrument of claim 15, and including an actuator mechanism operable to move the actuator relative to the handle and to the jaw members.

17. The instrument of claim 16, in which the actuator mechanism includes a rotatable driver coupled to the actuator and wherein rotation of the driver causes displacement of the actuator along the longitudinal axis of the instrument relative to the handle.

18. The instrument of claim 17, wherein the rotatable driver includes a first end face which is tilted relative to the longitudinal axis and the actuator includes a second end face which is parallel to the first end face and wherein the first and second end face abut to form the coupling between the rotatable driver and the actuator.

19. The instrument of claim 18, wherein the actuator includes an anti-rotation mechanism which prevents rotation of the actuator relative to the instrument
about the longitudinal axis but allows translation along the longitudinal axis.

20. An assembly for use in a surgical procedure to replace an orthopaedic joint, which comprises:
an instrument for positioning a cup component of an orthopaedic joint prosthesis, including an actuator and a plurality of jaw members arranged around a longitudinal axis of the instrument, each jaw member having:
a mounting end,
a gripping end with a transversely extending member which can be received in a recess in a wall of a cup component, wherein the gripping end of each of the jaw members can be displaced between a retracted position and a deployed position in which the gripping end is displaced from the retracted position in the direction in which the transversely extending member is directed, and
a surface between the mounting end and the gripping end which is inclined to the longitudinal axis of the instrument and curved in an outward direction away from the longitudinal axis, wherein the actuator and the plurality of jaw members are capable of relative movement along the longitudinal axis of the instrument with the actuator in contact with the inclined surfaces of the jaw members so that said relative movement causes the jaw members to move between their retracted position and deployed position; and
a cup component of an orthopaedic joint prosthesis, in which each of the generally transversely extending members on the jaw members of the instrument are received in a respective recess in a wall of the cup component.

21. The assembly of claim 20, wherein the gripping end of each jaw member includes a flat underside surface arranged to engage an upper surface of a rim of the cup component.
